(19) United States
(12) Patent Application Publication
(10) Pub. No.: US 2018/0304052 A1
(43) Pub. Date: Oct. 25, 2018

(54) MEDICAL BALLOON

(71) Applicant: Cagent Vascular, LLC, Wayne, PA (US)

(72) Inventors: Peter Schneider, Honolulu, HI (US); Robert M. Giasolli, Orange, CA (US)

(21) Appl. No.: 15/523,932

(22) PCT Filed: Nov. 3, 2015
(86) PCT No.: PCT/US2015/058874

§ 371 Date: May 2, 2017

Related U.S. Application Data
(60) Provisional application No. 62/074,548, filed on Nov. 3, 2014.

(51) Int. Cl. A61M 25/10

(52) U.S. Cl. CPC A61M 25/104; A61M 25/1002; A61M 2025/1004; A61M 2025/109; A61M 2025/1068; A61M 2025/1084; A61M 2025/105

(57) ABSTRACT

A medical balloon system can include a cage that can be used to control the outer diameter of the expanded balloon. This can be done to control features such as the maximum outer diameter, but also to better control drug exposure on the surface of the balloon.

### INCORPORATION BY REFERENCE TO ANY PRIORITY APPLICATIONS

This application claims priority to U.S. Provisional App. No. 62/074,548 filed Nov. 3, 2014. All of the above application(s) is/are incorporated by reference herein in their entirety and are to be considered a part of this specification. Any and all applications for which a foreign or domestic priority claim is identified in the Application Data Sheet as filed with the present application are hereby incorporated by reference under 37 CFR 1.57.

### BACKGROUND

### Field of the Invention

Certain embodiments disclosed herein relate generally to a medical balloon. Particular embodiments disclose features of a medical balloon such as an angioplasty balloon having adjustable outer dimensions, controlled cone angles, and methods of controlled tearing of plaque during an angioplasty procedure.

### Description of the Related Art

Atherosclerotic occlusive disease is the primary cause of stroke, heart attack, limb loss, and death in the United States and the industrialized world. Atherosclerotic plaque forms a layer along the wall of an artery and is comprised of calcium, cholesterol, compacted thrombus and cellular debris. As the atherosclerotic disease progresses, the blood supply intended to pass through a specific blood vessel is diminished or even prevented by the occlusive process. One of the most widely utilized methods of treating clinically significant atherosclerotic plaque is balloon angioplasty.

Balloon angioplasty is a method of opening blocked or narrowed blood vessels in the body. The balloon angioplasty catheter is placed into the artery from a remote access site that is created either percutaneously or through open exposure of the artery. The catheter is typically passed along the inside of the blood vessel over a wire that guides the way of the catheter. A portion of the catheter with a balloon attached is placed at the location of the atherosclerotic plaque that requires treatment. The balloon is inflated, generally to a size consistent with the original diameter of the artery prior to developing occlusive disease.

When the balloon is inflated, the plaque may be stretched, compressed, fractured, and/or broken, depending on its composition, location, and the amount of pressure exerted by the balloon. Plaque can be heterogeneous and may be soft in some areas or hard in others, causing unpredictable cleavage planes to form under standard balloon angioplasty. Balloon angioplasty can cause plaque disruption and sometimes arterial injury at the angioplasty site. There is a continuing need to improve the methods and systems for treating occlusive disease, including balloon angioplasty methods and systems.

### SUMMARY OF THE INVENTION

According to some embodiments, a medical balloon system can have an adjustable outer diameter. The medical balloon system can comprise an elongated shaft defining a longitudinal axis; a medical balloon on a distal end of the elongated shaft; and a control system. The control system can have a plurality of longitudinally extending members, each of the longitudinally extending members positioned along a surface of the medical balloon, the plurality of longitudinally extending members having at least two positions with respect to the elongated shaft to thereby control an outer diameter of the medical balloon. In at least one of the at least two positions when the medical balloon is in an expanded state the medical balloon can comprise a plurality of lobes, each lobe of the plurality of lobes being formed because of and between two longitudinally extending members of the plurality of longitudinally extending members; and the position of the plurality of longitudinally extending members with respect to the elongated shaft can control the maximum outer diameter of the medical balloon when the medical balloon is expanded.

The balloon can be a drug eluting balloon. In addition, each of the longitudinally extending members of the plurality of longitudinally extending members can comprise a plurality of protrusions configured to serrate plaque in a blood vessel.

A treatment method can include a number of steps such as: 1) advancing a medical balloon to a treatment location in a vessel having a narrowed diameter, the medical balloon having a cage positioned on an outside surface of the medical balloon, the cage and medical balloon both being in a collapsed state; 2) expanding the cage from the collapsed state to a first expanded state to serrate plaque at the treatment location, the cage having a plurality of longitudinally extending members each having protrusions located along a length of the longitudinally extending member, the protrusions configured to serrate plaque; 3) partially collapsing the cage to limit a maximum outer diameter of the medical balloon; and 4) expanding the medical balloon at the treatment location to expand the vessel, the expansion being limited by the cage and thereby creating lobes of the medical balloon on either side of each of the plurality of longitudinally extending members of the cage.

In some treatment methods expanding the medical balloon can further comprise exposing a drug coating on the medical balloon that can been positioned in folds in the balloon adjacent the longitudinal extending members.

A medical balloon system can provide controlled drug delivery to a vessel. Embodiments of the medical balloon system can comprise an elongated shaft defining a longitudinal axis; a medical balloon on a distal end of the elongated shaft; a plurality of longitudinally extending members, each of the longitudinally extending members positioned along a surface of the medical balloon; and a drug coating positioned on only select areas of an outer surface of the medical balloon. In a first state the medical balloon can comprise a first plurality of lobes, the balloon having a first outer surface and folds that create the lobes, the drug coating being completely positioned within the folds in the first state and thereby not being exposed to fluid flow in a vessel, each of the longitudinally extending members positioned along the first outer surface. In a second state the medical balloon is expanded from the first state and the medical balloon can comprise a second plurality of lobes wherein the drug coating in the folds of the first plurality of lobes now defines at least a portion of a second outer surface and the first outer surface of the first plurality of lobes is inward from the second outer surface, each lobe of the second plurality of lobes being formed because of and between two longitudinally extending members of the plurality of longitudinally extending members.

In some embodiments, the medical balloon can further comprise an adhesive that seals the folds of the first plurality of lobes to prevent premature exposure of the drug coating. Further, each of the longitudinally extending members can have protrusions located along a length of the longitudinally extending member, the protrusions configured to serrate plaque.

Another treatment method can include the steps of: 1) advancing a medical balloon to a treatment location in a vessel having a narrowed diameter, the medical balloon having a cage positioned along a surface of the medical balloon, the cage and medical balloon both being in a collapsed state, the cage having a plurality of longitudinally extending members; 2) expanding the medical balloon to a first state wherein the medical balloon comprises a first plurality of lobes, the balloon having a first outer surface and folds that create the lobes, a drug coating positioned within the folds in the first state and thereby not being exposed to fluid flow in the vessel, each of the longitudinally extending members positioned along the first outer surface; 3) expanding the medical balloon to a second state larger than the first wherein the medical balloon comprises a second plurality of lobes wherein the drug coating in the folds of the first plurality of lobes defines at least a portion of a second outer surface and the first outer surface of the first plurality of lobes is inward from the second outer surface, each lobe of the second plurality of lobes being formed because of and between two longitudinally extending members of the plurality of longitudinally extending members; and 4) exposing the treatment location in the vessel to the drug coating.

### BRIEF DESCRIPTION OF THE DRAWINGS

Various embodiments are depicted in the accompanying drawings for illustrative purposes, and should in no way be interpreted as limiting the scope of the inventions, in which like reference characters denote corresponding features consistently throughout similar embodiments.

FIG. 1 is a perspective view of a medical balloon system.

FIGS. 2A and 2C show perspective and end views respectively, of the cage of the medical balloon system of FIG. 1 in a collapsed state.

FIGS. 2B and 2D show perspective and end views respectively, of the cage of the medical balloon system of FIG. 1 in an expanded state.

FIG. 3 is a sectional view of the medical balloon system of FIG. 1.

FIG. 4 illustrates the medical balloon system in a state expanded from FIG. 1.

FIG. 5 is a sectional view of the medical balloon system further expanded from the state of FIG. 4.

FIGS. 6A and 6B are cross-sectional end views of FIGS. 3 and 4, respectively.

FIG. 7 shows a control system for a cage.

FIG. 8 illustrates a control system having a bi-directional screw.

FIG. 9 shows a webbing based control system.

FIGS. 10 and 11 show various relationships between webbing and linear filaments.

FIG. 12 shows a method of securing a linear filament.

FIG. 13 illustrates a balloon with a groove and a linear filament positioned in the groove.

FIG. 14 is another embodiment of a medical balloon system.

FIGS. 15A and 15B show a medical balloon system with a drug on selected portions of the balloon surface.

### DESCRIPTION

Disclosed herein are various embodiments of systems and methods discussed primarily in the context of treating occlusive disease, including balloon angioplasty methods and systems. At the same time, it will be understood that the concepts and principles embodied in the various embodiments can also be used with other types of medical balloons and other types of medical procedures.

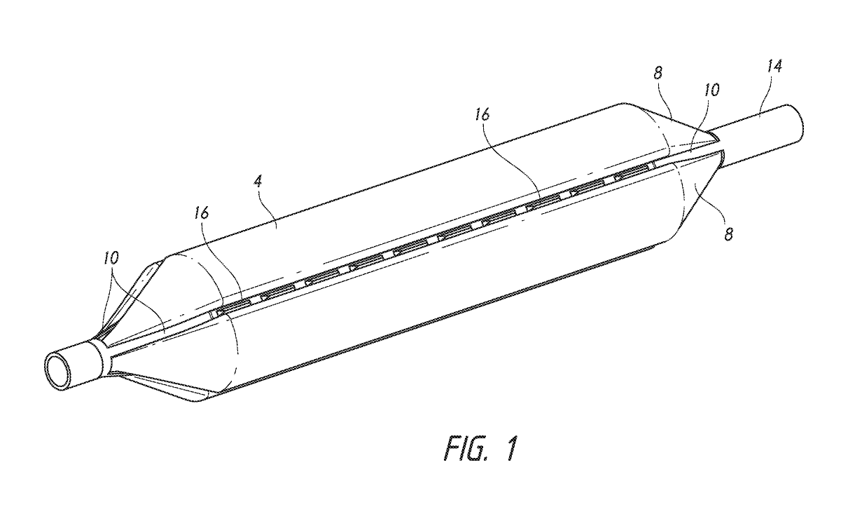

FIG. 1 shows a medical balloon system 100 including a catheter 2, a balloon 4 and a cage 6. The balloon can be attached to the catheter and the cage can be positioned on the outside of the balloon. The balloon 4 is shown in a first inflated condition with a first controlled outer diameter. The balloon 4 has a number of lobes 8 that, when deflated can be wrapped around the catheter 2. The illustrated embodiment has four lobes 8, though it will be understood that the balloon 4 can be folded and/or formed with any number of lobes. In addition, the lobes 8 can be physical portions of the balloon 4 that are formed with distinct shapes, or they can be created merely by folding an otherwise circular cross-sectioned balloon 4. An off-the-shelf or a custom balloon can be used. For example, an off-the-shelf percutaneous transluminal angioplasty (“PTA”) balloon can be used.

An off-the-shelf medical balloon, such as an angioplasty balloon can be used to create a serration or cutting balloon. The catheter balloon can have a catheter shaft with a balloon at the distal end. Radiopaque markers can be positioned inside the balloon. The shaft can be hollow and can be used to inflate the balloon and can also be used with a guidewire. Thus, the shaft can have two channels, one for inflation and one for positioning with a guidewire. A hub can be used with two entry points for the shaft and can be a y-hub and strain relief.

In some embodiments the catheter can be a coaxial over-the-wire balloon catheter with a guidewire size compatibility of 0.018″. A high pressure (non-compliant/semi-compliant) trifold balloon can be made of nylon material with a diameter of 5 mm and a length of 20 mm±1 mm. The balloon has a nominal inflation pressure of 10 atm. a rated burst pressure of 22 atm, and an average burst pressure of 22 atm. The catheter working length is 110 cm±2 cm and has a tapered tip length of 3 mm±0.5 mm. Two standard radiopaque makers 42 are made up of 90% platinum and 10% iridium. The radiopaque markers 42 indicate the balloon working length. The inner shaft has a lubricious HDPE inner layer. The profile of the outer shaft is clear and 4.3 FR (0.056 in ±0.0008 in; 1.42 mm±0.0200 mm.

The cage 6 can be a control system that limits an outer diameter on the balloon. The cage 6 can be positioned on the outside of the balloon 4 to restrict the balloon's ability to expand. The cage 6 can be adjustable to limit expansion of the balloon in a stepwise or an infinitely adjustable manner within a certain range. In some embodiments, the cage 6 for a PTA balloon can enable balloon diameter ranges above 1 mm. In some embodiments, the cage 6 can limit expansion of the balloon independent of the pressure within the balloon. This system can offer clinicians a new range of PTA dimensions with a single device.

A cage 6 can be positioned around the balloon 4. As shown, the cage 6 is positioned outside of the balloon 4, though in some embodiments the cage 6 can be positioned inside the balloon. In some embodiments the cage 6 can be positioned between two layers of material that form the balloon. The cage 6 can include a series of longitudinally extending members 10. The longitudinally extending members 10 can be in the form of strips 10 (shown in FIG. 1), wires, ribbons, fibers, splines, etc. The longitudinally extending members 10 are connected at both ends of the balloon 4. In some embodiments, the longitudinally extending members 10 have a number of protrusions 16 located along the length of the longitudinally extending members 10. As shown in FIG. 1, in one embodiment, these protrusions 16 can be spiked in shape. In other embodiments, the protrusions 16 can take a number of other shapes and can be variably spaced across the length of the longitudinally extending members 10. In some embodiment, the protrusions 16 are placed only on the cone or angled area of the longitudinally extending members 10, for example, only on one side (proximal and/or distal side).

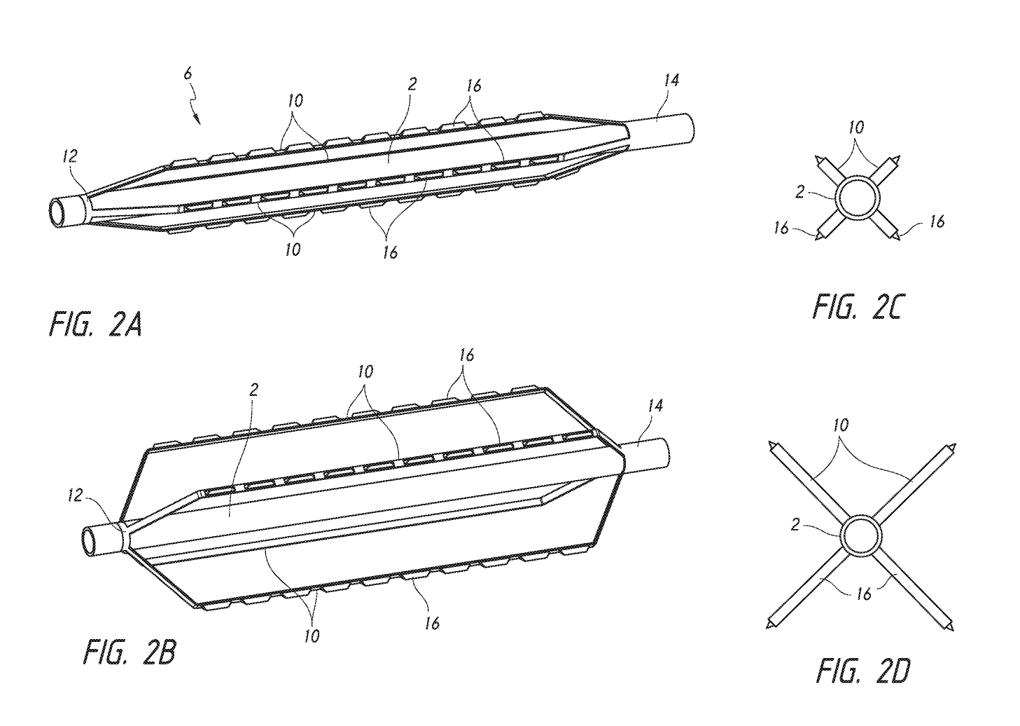

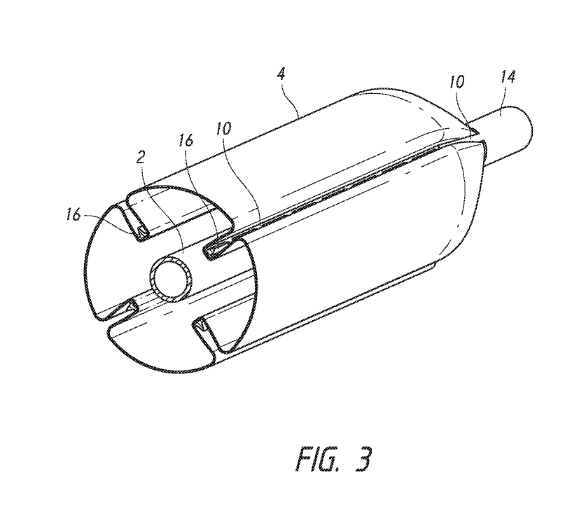

The cage 6 can be controlled by changing the linear distance between its two ends 12, 14 as shown in FIGS. 2A-D. One end of each of the longitudinally extending members 10 can be secured to a band 12 that is secured in position with respect to an elongate tube portion of the catheter 2. The opposite end of each longitudinally extending member 10 can be connected to a band 14. The band 14 can be part of or connected to a sheath or other movable member that can move with respect to the elongate tube of the catheter 2. As can be seen in FIGS. 2A and 2C, the cage 6 is in a first position (the balloon 4 has been removed to facilitate the understanding of the cage 6). The first position of the cage 6 is only slightly larger than the catheter 2. In the second position as shown in FIGS. 2B and 2D, band 14 has been moved towards the opposite end and towards band 12. This narrowing of the space between the ends decreases the length of the cage 6, while also increasing its outer diameter. This in turn, allows the balloon 4 to expand to a larger outer diameter.

As can be seen, in one embodiment, the longitudinally extending members 10 have been formed so as to have angled sections on either end connected by straight portions. A bend at the junction between the angled section and the straight section can be formed into the longitudinally extending members 10 to encourage them to take on this shape. Pre-forming the longitudinally extending members 10 can help control expansion of the balloon 4 in a particular manner. Other features such as material thickness and shape can also be used to control the expanded shape of the longitudinally extending members 10. As seen in FIG. 1, protrusions 16 can be located on the straight portions of the longitudinally extending members 10 and not on the angled sections. In other embodiments, the angled and straight sections may both include protrusions, though they may have different shapes and/or patterns.

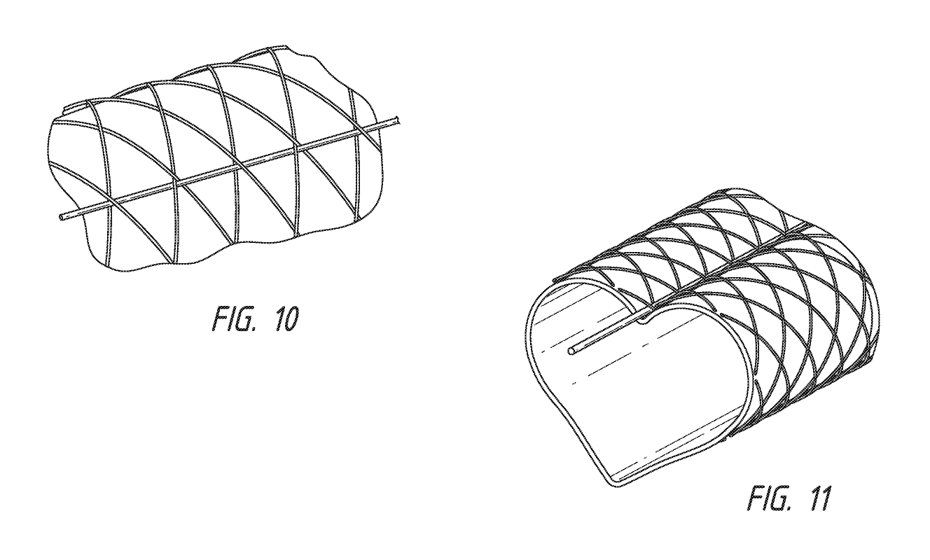

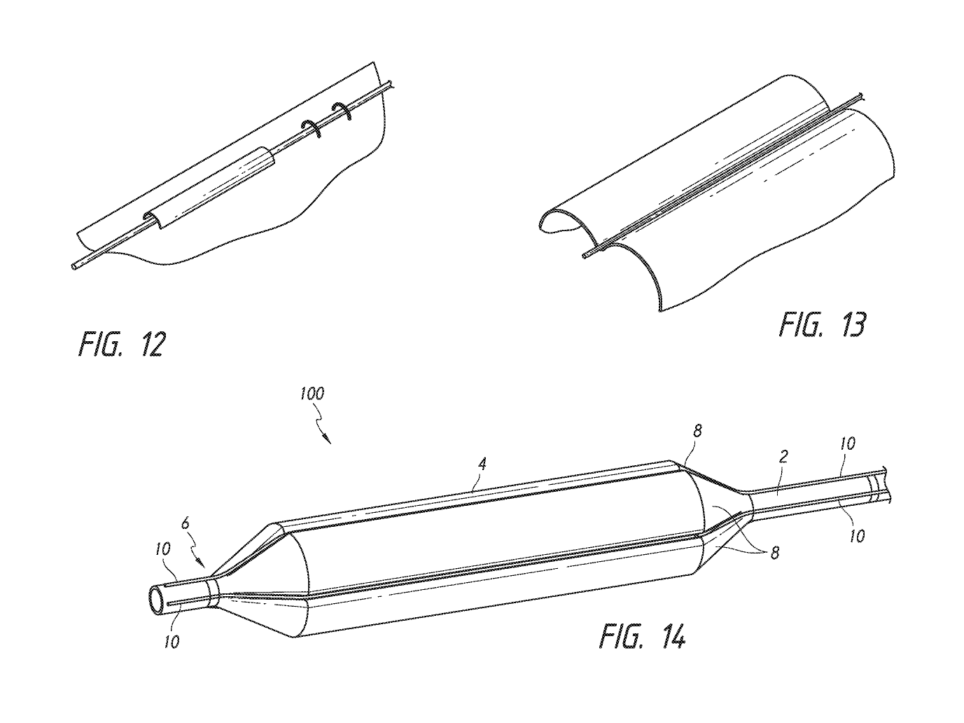

The longitudinally extending members 10 can be free floating with respect to the balloon 4, or can be attached in part or in whole to the balloon 4. For example, in one embodiment all or part of the distal angled portion of one or more of the longitudinally extending members 10 can be connected to a distal portion of the balloon 4 such as a cone shaped portion of the balloon 4. In some embodiments the longitudinally extending members 10 can be connected to the balloon 4 but able to slide or move with respect thereto (see FIGS. 10 and 12). For example, each longitudinally extending member 10 can be positioned within a sleeve on the outside of the balloon 4.

Looking now to FIGS. 3-6B, the expansion limiting features will be described with respect to the balloon 4. In FIG. 3 a section of the balloon 4 is shown in the first inflated position of FIG. 1. Four lobes 8 can be seen. Between each lobe, the outer surface of the balloon 4 is folded inward effectively forming the lobes 8. Each longitudinally extending member 10 can be positioned between two adjacent lobes 8. In this way the longitudinally extending member 10 can restrict movement of the folded outer material, thereby restricting expansion of the balloon 4. As can be seen in FIG. 3, the balloon 4 is still allowed to expand, but the outer diameter is limited by the cage 6 positioned between the lobes 8.

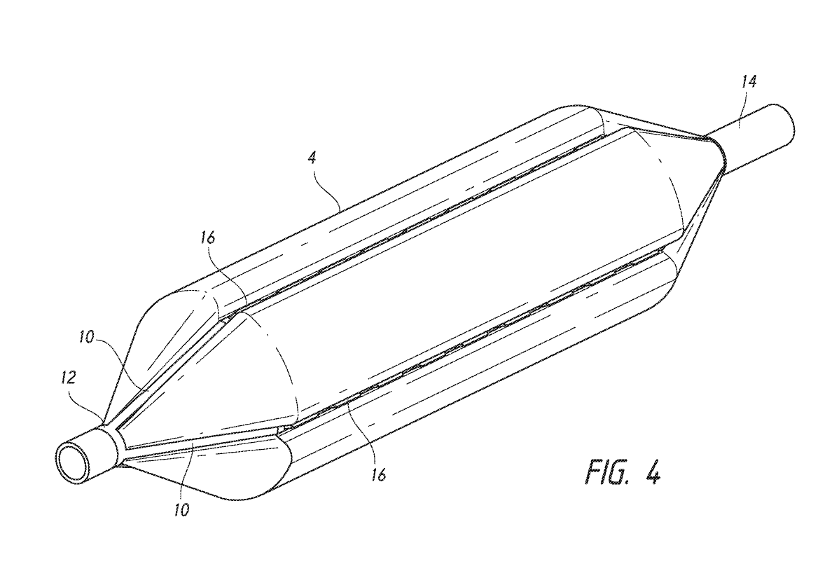

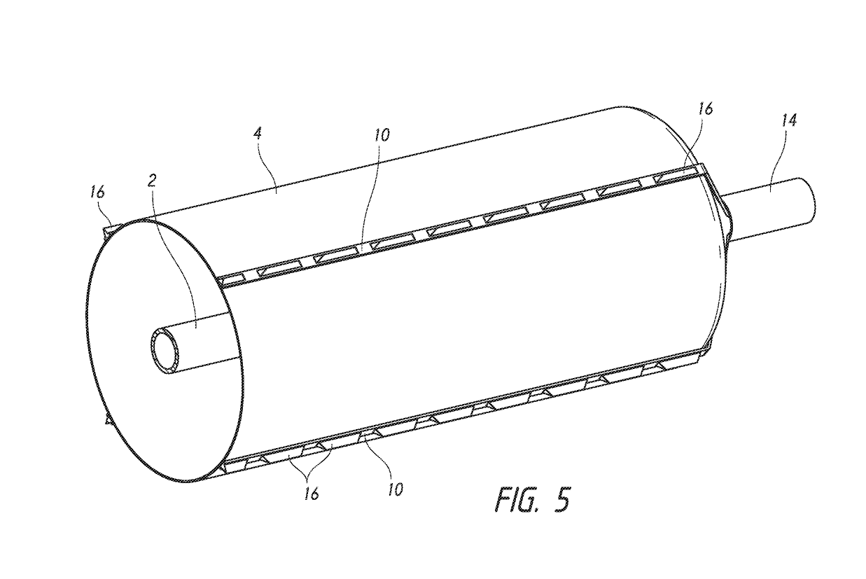

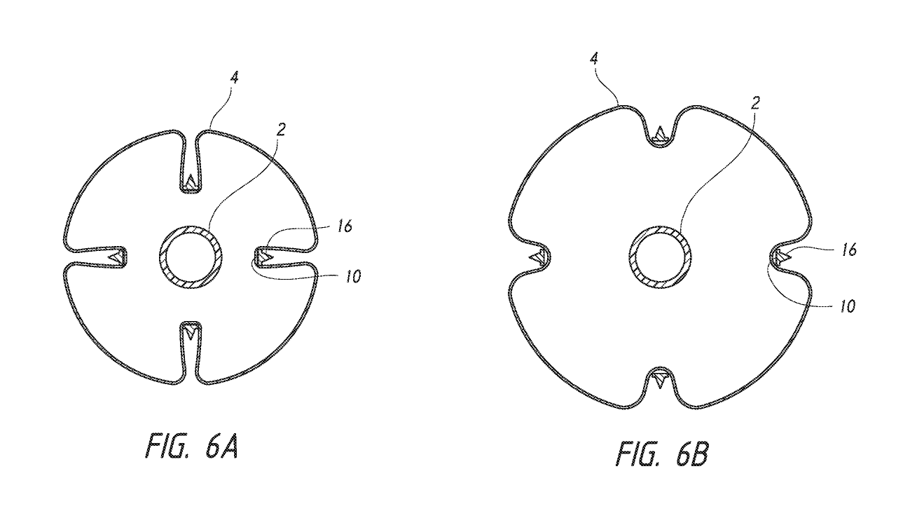

Increasing the diameter of the cage 6 by decreasing its length reduces the restrictions on movement of the outer material of the balloon 4. Thus, the outer material can expand further to increase the diameter of the balloon 4. This can be seen in FIG. 4. FIG. 5 shows the balloon 4 expanded to its full extent. In this position, the cage 6 does not limit expansion of the balloon 4. Though, in some embodiments, it may be beneficial to provide some restriction on balloon expansion with the cage 6 over the entire range of desired expansion. FIGS. 6A and 6B are cross-sectional views of FIGS. 3 and 4, respectively.

In the illustrated embodiment, when the balloon 4 is expanded, an increasing amount of pressure (typically in a hydraulic form) expands the outer diameter of the balloon 4 until the point where the cage 6 constricts further expansion. Once the desired limit of expansion of the outer diameter of the cage 6 is obtained, the operator may continue to increase the pressure of the balloon 4 up to a desired pressure. Despite the increasing pressure of the balloon 4, the cage 6 can maintain a set outer diameter. The cage 6 thereby offers a unique ability to separate the outer diameter of the balloon 4 from the typical mechanism used to expand the balloon 4 (namely pressure). The mechanism of control over the outer diameter of the cage 6 can be done by moving one or both sides of the cage 6 towards each other. As a result of the expansion or constriction of the cage 6, the material of the balloon 4 that is taut or stretched is loosened. This allows for further expansion of the balloon 4. Among other features, the range of expansion depends in large part on the size, orientation, and number of longitudinally extending members 10 that restrict the balloon 4. In some embodiments the balloon is restricted by 5 linear wires oriented longitudinally across the balloon surface and the wires are thin enough to be relatively non-obtrusive. Other types of longitudinally extending members can also be used. As an example: the diameter of a balloon can be controlled within a range of 1.125-6.000 mm, 1.50-6.00 mm, 1.75-6.0 mm, 2.0-6.0 mm. etc. for a 6 mm outer diameter balloon. The cage 6 can control the diameter of the balloon 4 up to and within a range of 3-4 times, 2-5 times, or more from the initial expanded position to the fully expanded position. In some embodiments the range can be adjusted with accuracies of tenths to thousands of a mm.

Typically, the distal end of the cage 6 will be fixed, bonded, sealed, braided, wrapped, or crimped to the balloon 4 carrying catheter. The other end of the cage can be attached, bonded, sealed, braided, wrapped, or crimped to a functional component, such as the band 14 discussed above. In some embodiments, the functional component can be precisely positioned relative to the fixed end. As the position of the functional component is shifted towards the fixed side, the longitudinally extending members 10 expand outward. In its initial position, the longitudinally extending members 10 are stretched and lay in a generally flat or parallel configuration on the outer most surface of the balloon. The initial position of the longitudinally extending members 10 can be subject to many factors including the thickness of the balloon material.

In some embodiments, as the longitudinally extending members 10 expand, they bow outward towards the wall of the blood vessel. In some embodiments, moving the two ends of the cage closer together releases tension on the longitudinally extending members 10. Expansion of the balloon then enables the functional diameter of the balloon to increase.

As has been mentioned, the longitudinally extending members 10 can be positioned in the creases of the folds of the balloon when the balloon is in an expanded state. This provides the balloon with the ability to expand uniformly in the areas between the longitudinally extending members 10 and limits the energy imparted on the longitudinally extending members 10 when the longitudinally extending members 10 are in the fully expanded state.

In some embodiments, the longitudinally extending members 10 can be positioned completely outside the balloon 4 and can define an outer diameter of the device. As the balloon expands it may expand into the cage 6 or with the cage 6. In such embodiments, there preferably would not be separate lobes, but rather the balloon as whole would expand within the cage to the outer limit defined by the cage.

The controlled and staged expansion of the cage 6 can also provide for the controlled and uniform expansion of the balloon 4 along its length. A problem frequently faced in balloon angioplasty is the uneven expansion of the angioplasty balloon—termed “dog boning.” Dog boning occurs when, at the treatment site, the proximal and distal ends of the balloon expand more than the center of the balloon—likened to a dog bone. Dog boning reduces the effectiveness of balloon angioplasty as it reduces the effective diameter of the balloon 4 at the site of treatment. The structure of the cage 6 can help ensure the uniform expansion of the balloon 4 along its length. Further, the cage 6 provides the ability to incrementally increase the diameter of the balloon 4 which, in turn, allows the treatment site to expand in an incremental and controlled manner.

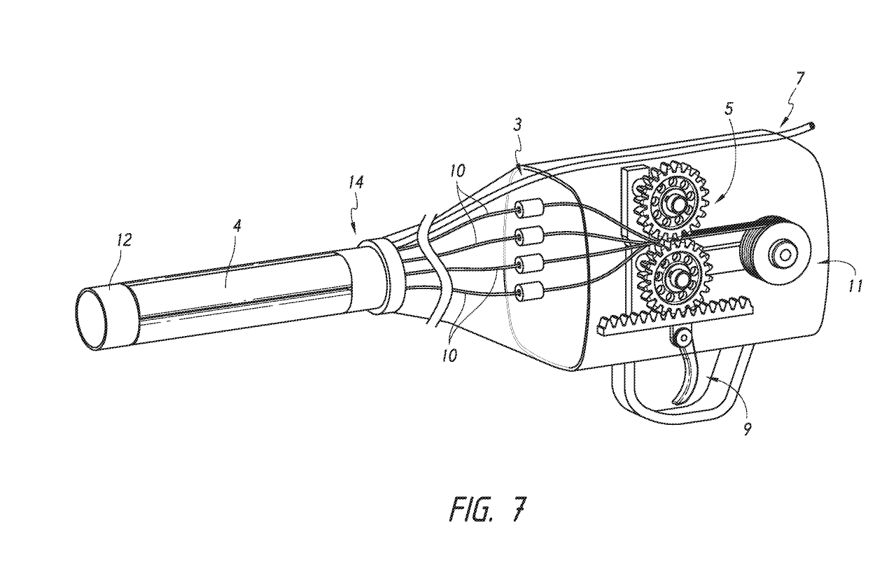

Turning now to FIG. 7, a control system for a cage 6 is shown. The control system can be used to effectively manage an accurate and precise length of the cage to thereby accurately control balloon expansion. The control system can include one or more wires or other filaments 10 connected to the proximal hub 14 of the cage 6 at one end to control the position of the proximal hub 14 with respect to the distal hub 12. In some embodiments, the distal hub 12 can be fixed in position, while the proximal hub position can be adjusted. For example, the proximal hub 14 can be capable of moving proximally by retraction of the filaments. Depending on the pushability of the filament 10, the proximal hub 14 can move distally by release or distal advancement of the filament, which may also need to be done in combination with inflation of the balloon. Each filament can run through a lumen, which lumen or series of lumens can surround one, two, or more central lumens. An operator control mechanism (OCM) 9 can be used to control the position of the filaments. The OCM can be positioned at the proximal end of the catheter and can be used to adjust the position of each filament individually and/or the filaments collectively.

In some embodiments, some of the filaments may also form the longitudinally extending members 10 of the cage 6. These longitudinally extending members 10 may be fixed with respect to the proximal hub or adjustable. In this way, the working length of the cage 6 and therefor of the balloon can be adjusted independent of the pressure of the balloon.

In some embodiments, the catheter is packaged with a pre-determined length of filament. This can preferably include a small amount of extra filament to provide enough length so as to not bind the balloon as the catheter migrates through the anatomy. In use within the body, the catheter may encounter various anatomical tortuosities. Once the catheter is positioned at the treatment location, the operator control mechanism (OCM) may be manipulated by the operator. In some embodiments, the OCM can be used initially to tighten each individual filament relative to the proximal hub and/or distal hub of the cage, depending on the embodiment. This tightening allows the system to accommcidate for the unknown tortuosity of the vessel. The filaments can be adjusted individually and/or collectively once the system is in the desired location. This allows the system to adapt to conditions where one filament is on the inside of the small radius of curvature while other filaments have a slightly larger radius of curvature. There can be a one to one correlation between the operator control mechanism (OCM) and the proximal and/or distal hub of the cage. When the OCM is tightened, the filaments can limit the balloon diameter. In addition, if some of the filaments cross the balloon to collectively form the cage, this may also limit the balloon diameter and/or length. In contrast, loosening the OCM and inflating the balloon, loosens the filaments allowing for controlled expansion to larger balloon diameters and/or lengths.

In FIG. 7, the operator control mechanism (OCM) is shown with a trigger connected to a rack and pinion 5. A spool 11 is also shown around which the extra filament is wrapped. Movement of the trigger 9 can cause the filament to loosen or tighten, depending on the direction of movement. Individual tabs 3 are shown associated with each filament. These tabs can be adjusted to increase or decrease tension on the individual filament. For example, the height of the tab can be increased or decreased. A hub 7 can be used with one or more entry points to the catheter shaft to inflate the balloon and/or provide a channel for a guidewire.

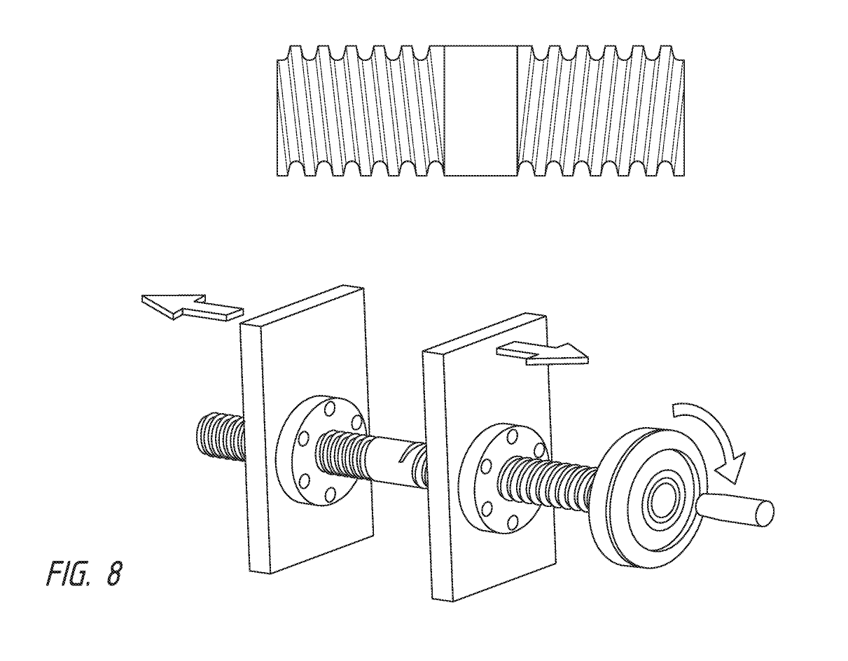

FIG. 8 illustrates another control system having a bi-directional lead screw platform. The bi-directional lead screw can run at least the length of the balloon and be attached to both the proximal and distal hubs of the cage. The screw can be controlled by a hollow catheter that runs to the OCM. The OCM, represented schematically by the handle in the figure, can be rotated either through a gear like mechanism, series of gears, or directly at the OCM. This rotation can rotate the hollow shaft that runs the length of the catheter and in turn rotates the bi-directional lead screw. The direction of rotation of the bi-directional lead screw determined whether both the proximal and distal hubs are pushed away from or towards each other. As the hubs move, the cage is tightened or loosened which translates into the dynamic balloon diameter control.

Independent of the control system and OCM used, the balloon diameter can be allowed to expand over a range of diameters with a predetermined rate of expansion. This can be done by controlling tension in the OCM to allow for slow or predetermined rates of expansion until a set point of balloon diameter is reached.

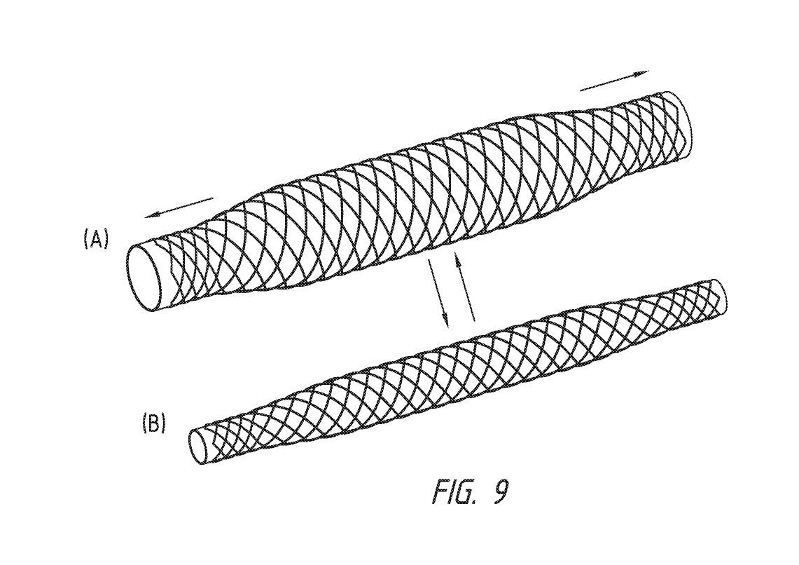

FIG. 9 illustrates another system that can limit or control the outer diameter of the balloon. A web is shown over the balloon in expanded and collapsed positions. The web can be applied to the balloon with the balloon in the collapsed position. For example, the balloon can be folded in the collapsed position and the web can then be applied to the balloon. In other embodiments, the web can be applied to the balloon in the expanded position and then collapsed and possibly folded. The tightness of the web can determine the maximum outer diameter of the balloon. The web can be weaved fibers which fibers can be tightened by an OCM in a manner similar to those previously described. For example, the web can be connected to proximal and distal hubs. In addition, the fibers can be directly connected to the OCM as previously described with respect to the filaments.

In addition, or alternatively, as shown in FIG. 10, the web can be used as a stabilizer for linear filaments that run along the surface of the balloon. The linear filaments may function as the cage while the weave may offer filament control. The linear filaments may also include a plurality of spikes to serrate plaque. In some embodiments, the linear filaments can move distally and proximally within the web.

In an alternate configuration shown in FIG. 11, the webbing has some areas bound to the balloon and other areas not bound. The non-bounded sections have room for a longitudinal filament to be threaded along and under some or all of the web material. This can control the orientation of the filaments while allowing it to move freely. As the balloon folds, the web can collapse with the balloon. The web can be folded into the lobes created by the filaments (longitudinally extending members 10 as in FIGS. 1-6B). As also shown in FIG. 11, the balloon can be designed to expand primarily in one direction, or a number of segmented balloons can be attached to create a full 360 degree expansion. One segment is shown that could be combined with three, four or five other segments with each segment attached to the catheter shaft at the bottom of the heart shape shown.

Turning now to FIG. 12, additional types of systems for securing longitudinal filaments or longitudinally extending members to the balloon can be seen. These filaments can be channeled across the outer surface of the balloon by an eyelet or other hole, channel, or tube. FIG. 12 shows a filament passing through a tube and two eyelets on the outer surface of a balloon. The eyelet offers a mechanism to limit the shifting of the filament during relaxation and contraction of the filaments, for example, to prevent cork screwing around the balloon. The eyelet may be made of the same material as the balloon, plastic or metal material. The eyelet can be used to manage the filament in such a way as to maintain a consistent orientation and close profile to the balloon. The eyelets can be generated during the molding of the balloon or after the balloon molding process. They can be put in place with glue, wire or fiber wrapping, thermal, compression or ultrasonic bonding, or by some other mechanism that integrates an eyelet or channel within or on the outside of the surface of the balloon. The eyelets can be spaced apart uniformly in rows or each row can be offset from each other so as to allow for more effective balloon collapse and folding.

In other embodiments, the filaments can run along groves molded into the surface of the balloon (one representative groove and filament shown in FIG. 13). For example, the grooves can be equally spaced apart around the balloon. Groves may or may not run along the entire surface of the balloon and may not bind the filament but instead can limit the tendency of the filaments from shifting randomly along the outer surface of the balloon.

In some embodiments the balloon can be a drug eluting balloon (“DEB”). The DEB can have longitudinally extending members 10 positioned between the folds of the balloon 4 creating lobes 8 (FIG. 14). The lobes 8 can also be used to limit exposure of the drug coating on the outer surface of the balloon 4. For example, the drug coating can be positioned within the folds between the lobes 8 (in other words, within the crevice created by the longitudinally extending members 10). This portion of the drug coating can be prevented from being exposed or rubbed against by the vessel until after initial expansion of the balloon 4. In some embodiments the DEB can include a drug coating only within the folds of the balloon 4. In some embodiments, the drug coating can be applied only in select areas. In some embodiments drug coatings may be found in only a portion of the folded area. In the illustrated embodiment, the longitudinally extending members 10 are shown as wires which because of their small size can allow the lobes to more easily be positioned adjacent one another without, or with minimal gaps.

In some embodiments, the lobes can be secured together, such as with adhesive to further prevent the drug coating from becoming prematurely exposed to the vessel. Expansion of the balloon can break the seal created by the adhesive to then treat the desired area with the drug.

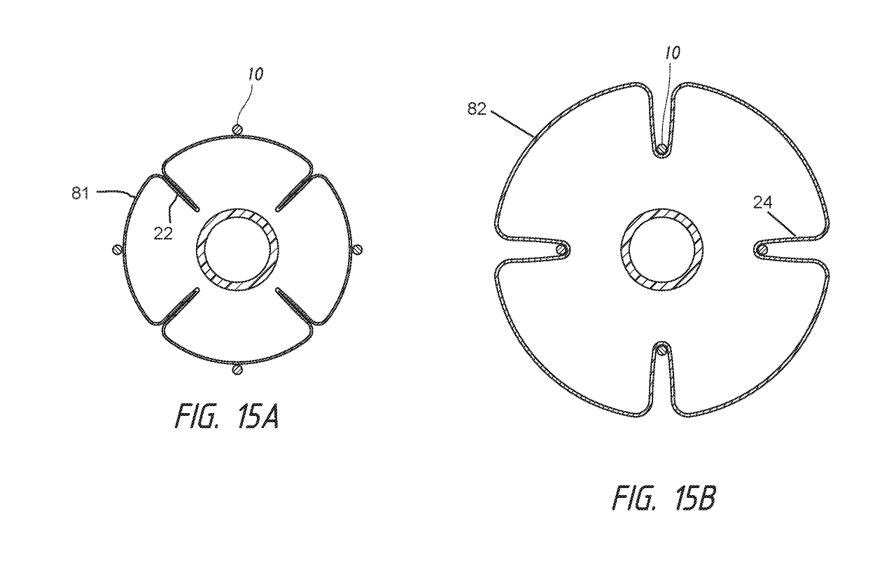

In some embodiments the longitudinally extending members 10 are positioned on the outside of the folds 22 of the balloon 4 (FIG. 15A) and may be located slightly projected from the surface of the balloon 4. This positioning of the longitudinally extending members 10 can be used to limit premature exposure of the drug coating on the outer surface of the balloon positioned within the folds 22. For example, the outer surface can form a number of first lobes 81. Preferably, the outer surface of the first lobes does not include drug coating. But the surface of the balloon in the folds 22 does include a drug coating. This drug coating can be protected, limiting exposure or rubbing of the drug coating within the folded sections 22 of the balloon 4 until after initial expansion of the balloon 4. During expansion (FIG. 15B) of the balloon 4, the position of the longitudinal extending members 10 can remain constant so that the folded sections 22 of the balloon unfold, and the previously exposed sections 24 fold up creating new lobes 82. This exposes the drug coating on the new lobes 82 (FIG. 15B) that was previously positioned within the fold 22 (FIG. 15A). In addition, the outer surface and the longitudinal members can be positioned to limit the amount of outer surface that does not include drug coating in the expanded position. New folded areas 24 are formed with little to no drug coating. The previously retained surfaces 22 form a new lobe 82 with the uncoated surface from the prior lobes 81 within the newly formed crease 24 and allowing exposure of the drug coated section of the balloon on new the outer surface of the balloon. This allows the amount of drug coating to be minimized and allows for more predictable delivery of drug at the desired treatment site. In some embodiments the DEB can include a drug coating only within the folds 22 of the balloon.

Once the DEB has reached the diseased site, the balloon 4 can be inflated to a diameter that is less than the diameter of the surface of the disease and then slowly inflated to the desired diameter. As the balloon 4 inflates beyond the initial diameter, the drug coating can become exposed and can be effectively delivered to the diseased site. By limiting the surface area of the balloon 4 with drug coating, the cage 6 can enable a greater level of control and drug retention until a point in time when release of the drug through contact is desired.

In some embodiments, the protrusions 16 are provided with a drug coating. Similarly, in some embodiments, the longitudinal extending members 10 are provided with a drug coating.

For the drug coated section, the following approach to coating of a balloon may be used. The surface of the balloon can be altered to produce a surface roughness or topographic match to the drug with predetermined, controlled and optimized geometries. The known geometry or roughness is uniquely designed to match the drug coding. The method used to enhance the surface roughness can be either additive or subtractive in nature, such as Nano-technology structures coated where desired or oblate from the surface or move materials around the surface using technology such as ultrasonics. This design offers a unique advantage to drug coatings such as limiting or reduce drug dilution or sloughing off as the balloon moves through a tortuous anatomy to the site of disease. The surface can also be optimized to enable sections of the balloon to have high drug adhesion like properties and other sections to have poor or low drug adhesion like properties. Therefore sections can be designed as drug-phobic and other sections to be drug-philic. When dipped sprayed or otherwise coated with drugs the balloon is quadrantly drug coated by design.

In some embodiments, in addition to controlling the diameter of the balloon 4, the medical balloon system 100 can also control the length of the balloon 4. For example, an outer sheath can be used to control the exposed balloon length, and the sheath can prevent the remainder of the balloon 4 from expanding. In some embodiments, the cage 6 can be constructed of a shape memory alloy with tension wires attached to band 14. In this embodiment, release of individual tension wires can allow for expansion of the cage 6 to a predetermined outer diameter.

According to some embodiments, a medical balloon system 100 can include a control system or cage 6 to control an adjustable outer diameter of the balloon 4. The control system can be pressure independent and can provide a stepped diameter or a continuously variable diameter within a set range. The balloon 4 can be a single balloon or a single chamber balloon, though multiple balloons or multiple chamber balloons can also be used. In some embodiments the length of the balloon 4 can also be controlled, such as with a stiff outer sheath. The cage 6 can be an outer wire frame that limits expansion of the balloon 4.

In some embodiments the balloon 4 can move between different star shaped cross sections until achieving a final fully expanded cross section. The final or intermediate cross section may be star shaped or circular. The balloon 4 can be formed in other shapes and configurations as well. In some embodiments, spikes can be positioned on the longitudinally extending members of the cage 6 between lobes 8 of the balloon 4.

Another benefit of the controlled balloon expansion system is it can allow for control of the angle of energy departed to the surface of the body lumen. According to some embodiments, this may be achieved through control of the depth of longitudinally extending members or the diameter at which the constrained balloon makes contact with the lumen wall. With a controlled depth of the longitudinally extending members, an angular depression can be generated along the lumen axis of the balloon that can apply a tangential force against the lumen wall at an angle of 45 degrees or less perpendicular to the lumen axis. At this angle the lumen tissue is susceptible to separating along the mid line of the depressed region. It can be noted that when attempting to tear a 2-D surface it is observed that an angle less than 90 degrees exists and offers greater control for predetermining the tear location and reduces the energy required to start and facilitate the continuation of a tear in the 2-D surface of many materials. When inducing expansion of arteries or other lumen tissue it is observed that the angle of energy departed at the lumen surface has an expansion effect at a similar angle to that as observed in the 2-D surface example. It has been observed that angles equal to or less than 45 degrees appear to have beneficial tearing effects on plaque in a blood vessel, although other predetermined angles may be used when tissue expansion is not the only desired effect.

First, the depth of the longitudinal extending members 10 can be set to optimize the angle or tangential energy for the tissue interface with the balloon 4. Next, the combination of the balloon 4 and the longitudinal extending members 10 is placed in the area for desired dilation and pressure is increased in the system. The combination of the balloon 4 and the longitudinal extending members 10 contacts the wall of the vessel and slowly the tension on the array of longitudinal extending members 10 is released. As the pressure is released, slight expansion of the balloon diameter occurs and tends to depart energy against the wall of the vessel. Because the longitudinal extending members 10 restrain the balloon surface and thereby generates a series of linear depressions at each longitudinal extending members 10 that are optimally aligned with the lumen axis. The force induced by the balloon expansion which is surrounded by a cage 6 and longitudinal extending members 10 is not only radial but also has a perpendicular force that is lateral to the surface of the lumen. Optimally the design leverages the radial energy for expansion of the balloon 4 to induce a portion of the energy into a perpendicular energy that promotes an expansion of the diseased tissue along the axis of the longitudinal extending members 10. This perpendicular force has the tendency to encourage a gentler and less injurious expansion of the tissue while the radial force behaves like a compression force against the lumen wall.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

Similarly, this method of disclosure, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

What is claimed is:

1. A medical balloon system having an adjustable outer diameter comprising:
an elongated shaft defining a longitudinal axis;
a medical balloon on a distal end of the elongated shaft;
a control system having a plurality of longitudinally extending members, each of the longitudinally extending members positioned along a surface of the medical balloon, the plurality of longitudinally extending members having at least two positions with respect to the elongated shaft to thereby control an outer diameter of the medical balloon;
wherein in at least one of the at least two positions when the medical balloon is in an expanded state the medical balloon comprises a plurality of lobes, each lobe of the plurality of lobes being formed because of and between two longitudinally extending members of the plurality of longitudinally extending members; and
wherein the position of the plurality of longitudinally extending members with respect to the elongated shaft controls the maximum outer diameter of the medical balloon when the medical balloon is expanded.

2. The medical balloon of claim 1, wherein each of the longitudinally extending members of the plurality of longitudinally extending members comprises a plurality of protrusions configured to serrate plaque in a blood vessel.

3. The medical balloon of claim 1, wherein the balloon is a drug eluting balloon and the drug is positioned only on a portion of the outer surface of the balloon.

4. The medical balloon of claim 1, wherein a drug coating is only positioned within folds of the balloon between lobes when the balloon is partially inflated.

5. A treatment method comprising:
advancing a medical balloon to a treatment location in a vessel having a narrowed diameter, the medical balloon having a cage positioned on an outside surface of the medical balloon, the cage and medical balloon both being in a collapsed state;
expanding the cage from the collapsed state to a first expanded state to serrate plaque at the treatment location, the cage having a plurality of longitudinally extending members each having protrusions located along a length of the longitudinally extending member, the protrusions configured to serrate plaque;
partially collapsing the cage to limit a maximum outer diameter of the medical balloon;
expanding the medical balloon at the treatment location to expand the vessel, the expansion being limited by the cage and thereby creating lobes of the medical balloon on either side of each of the plurality of longitudinally extending members of the cage.

6. The treatment method of claim 5, wherein expanding the medical balloon further comprising exposing a drug coating on the medical balloon that can been positioned in folds in the balloon adjacent the longitudinal extending members.

7. A medical balloon system to provide controlled drug delivery to a vessel comprising:
an elongated shaft defining a longitudinal axis;
a medical balloon on a distal end of the elongated shaft;
a plurality of longitudinally extending members, each of the longitudinally extending members positioned along a surface of the medical balloon;
a drug coating positioned on only select areas of an outer surface of the medical balloon;
wherein in a first state the medical balloon comprises a first plurality of lobes, the balloon having a first outer surface and folds that create the lobes, the drug coating being completely positioned within the folds in the first state and thereby not being exposed to fluid flow in a vessel, each of the longitudinally extending members positioned along the first outer surface;
wherein in a second state the medical balloon is expanded from the first state and the medical balloon comprises a second plurality of lobes wherein the drug coating in the folds of the first plurality of lobes now defines at least a portion of a second outer surface and the first outer surface of the first plurality of lobes is inward from the second outer surface, each lobe of the second plurality of lobes being formed because of and between two longitudinally extending members of the plurality of longitudinally extending members.

8. The medical balloon of claim 7, further comprising an adhesive that seals the folds of the first plurality of lobes to prevent premature exposure of the drug coating.

9. The medical balloon of claim 7, wherein each of the longitudinally extending members having protrusions located along a length of the longitudinally extending member, the protrusions configured to serrate plaque.

10. A treatment method comprising:
advancing a medical balloon to a treatment location in a vessel having a narrowed diameter, the medical balloon having a cage positioned along a surface of the medical balloon, the cage and medical balloon both being in a collapsed state, the cage having a plurality of longitudinally extending members;
expanding the medical balloon to a first state wherein the medical balloon comprises a first plurality of lobes, the balloon having a first outer surface and folds that create the lobes, a drug coating positioned within the folds in the first state and thereby not being exposed to fluid flow in the vessel, each of the longitudinally extending members positioned along the first outer surface; and
expanding the medical balloon to a second state larger than the first wherein the medical balloon comprises a second plurality of lobes wherein the drug coating in the folds of the first plurality of lobes defines at least a portion of a second outer surface and the first outer surface of the first plurality of lobes is inward from the second outer surface, each lobe of the second plurality of lobes being formed because of and between two longitudinally extending members of the plurality of longitudinally extending members; and
exposing the treatment location in the vessel to the drug coating.

11. The method of claim 10, further comprising expanding the cage from the collapsed state to a first expanded state to serrate plaque at the treatment location, each of the longitudinally extending members having protrusions located along a length of the longitudinally extending member, the protrusions configured to serrate plaque.
